(19) United States
(12) Patent Application Publication
(10) Pub. No.: US 2025/0340813 A1
(43) Pub. Date: Nov. 6, 2025

(54) IMPELLER ASSEMBLY FOR A BIOPROCESSING SYSTEM

(71) Applicant: Global Life Sciences Solutions USA LLC, Marlborough, MA (US)

(72) Inventors: Sree Ramulu Bandaru, Marlborough, MA (US); Timothy Becker, Marlborough, MA (US); Ivan Reed, Portsmouth Hampshire (GB)

(73) Assignee: Global Life Sciences Solutions USA LLC, Marlborough, MA (US)

(21) Appl. No.: 18/654,079

(22) Filed: May 3, 2024

(51) Int. Cl. C12M 1/04; B01F 27/113; B01F 27/808; B01F 27/91; B01F 35/00; B01F 101/44; C12M 1/00

(52) U.S. Cl. CPC C12M 27/04; B01F 27/113; B01F 27/808; B01F 27/91; B01F 35/189; C12M 23/14; C12M 23/26; C12M 29/08; B01F 2101/44

(57) ABSTRACT

An impeller assembly for a bioprocessing system includes a hub, a plurality of blades disposed along a circumferential direction of the hub and spaced apart from each other, and at least one vent hole in the impeller assembly providing a pathway for gas to travel from an area beneath the impeller assembly to an area above the impeller assembly.

### BACKGROUND

### Technical Field

Embodiments of the invention relate generally to bioprocessing systems and methods and, more particularly, to an impeller assembly for a bioprocessing system.

### Discussion of Art

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

In the biopharmaceutical industry, single use or disposable containers are often used for bioprocessing operations. Such containers can be flexible or collapsible plastic bags that are supported by an outer rigid structure such as a stainless steel shell or vessel. Use of sterilized disposable bags eliminates time-consuming step of cleaning of the vessel and reduces the chance of contamination. The bag may be positioned within the rigid vessel and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the bag/vessel.

Depending on the fluid being processed, the bioreactor system may include a number of fluid lines and different sensors, probes and ports coupled with the bag for monitoring, analytics, sampling, and liquid transfer. For example, a harvest port is typically located at the bottom of the disposable bag and the vessel, and allows for a harvest line to be connected to the bag for harvesting and draining of the bag. In addition, existing bioreactor systems typically utilize spargers for introducing a controlled amount of a specific gas or combination of gases into the bag. A sparger outputs small gas bubbles into a liquid in order to agitate and/or dissolve the gas into the liquid, or for carbon dioxide stripping. The delivery of gas via spargers helps in mixing a substance, maintaining a homogenous environment throughout the interior of the bag, and is sometimes essential for growing cells in a bioreactor.

For example, during bioreactor cell culturing, oxygen is delivered to cell media by means of sparging oxygen bubbles into the cell media to allow for dissolved oxygen to enable cellular respiration. As indicated above, an impeller is used to assist in sparge bubble dispersion and mixing of the process fluid. Typically, these impellers are placed above the sparging elements so that the sparge gas is evenly distributed throughout the bioreactor. These bubbles rise and can become entrapped within cored out features of the impeller assembly, which can create localized nonhomogeneous zone within the bioreactor. Furthermore, sparge bubbles can pop when they reach the liquid/gas interface of this trapped gaseous zone. This popping of bubbles releases energy that can damage cells in close proximity.

In view of the above, there is a need for an impeller assembly for a bioprocessing system that minimizes the trapping of sparge gas bubbles.

### BRIEF DESCRIPTION

In an embodiment of the invention, an impeller assembly for a bioprocessing system is provided. The impeller assembly includes a hub, a plurality of blades disposed along a circumferential direction of the hub and spaced apart from each other, and at least one vent hole in the impeller assembly providing a pathway for gas to travel from an area beneath the impeller assembly to an area above the impeller assembly.

In another embodiment of the invention, a bioprocessing system is provided. The bioprocessing system includes a bioprocessing container, an impeller base plate affixed to a bottom of the bioprocessing container, and an impeller received on the base plate, the impeller including a hub, a plurality of blades disposed along a circumferential direction of the hub and spaced apart from each other, and at least one vent hole in the impeller providing a pathway for gas to travel from an area beneath the impeller to an area above the impeller.

In yet another embodiment of the invention, a method for bioprocessing is provided. The method includes the steps of agitating a fluid within a bioprocessing vessel via rotation of an impeller positioned within the bioprocessing vessel, providing sparge gas to the fluid within the bioprocessing vessel at a location generally beneath the impeller, and via vent holes within the impeller, allowing the sparge gas to pass through the impeller from a location generally beneath the impeller, to a location generally above the impeller.

### BRIEF DESCRIPTION OF DRAWINGS

The present invention will be better understood from reading the following description of non-limiting embodiments, with reference to the attached drawings, wherein below:

FIG. 1 is a front elevational view of a bioprocessing system according to an embodiment of the invention.

FIG. 2 is a simplified side elevational, cross-sectional view of the bioprocessing system of FIG. 1.

FIG. 3 is a bottom plan view of an impeller of the bioprocessing system of FIG. 1, according to an embodiment of the invention.

FIG. 4 is an enlarged, detail view of area A of FIG. 3.

FIG. 5 is an enlarged detail view of a portion of the impeller of FIG. 3.

FIG. 6 is a bottom, perspective view of a blade of a prior art impeller.

FIG. 7 is a top, plan view of a portion of a blade of the impeller of FIG. 3.

FIG. 8 is a bottom, perspective view of an impeller of the bioprocessing system of FIG. 1, according to another embodiment of the invention.

FIG. 9 is a top plan view of a portion of the impeller of FIG. 8.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems. As used herein, the term “bag” means a flexible or semi-rigid container or vessel used, for example, as a bioreactor or mixer for the contents within.

Embodiments of the invention provide an impeller assembly for a bioprocessing system. In an embodiment, an impeller assembly includes a hub, a plurality of blades disposed along a circumferential direction of the hub and spaced apart from each other, and at least one vent hole in the impeller assembly providing a pathway for gas to travel from an area beneath the impeller assembly to an area above the impeller assembly.

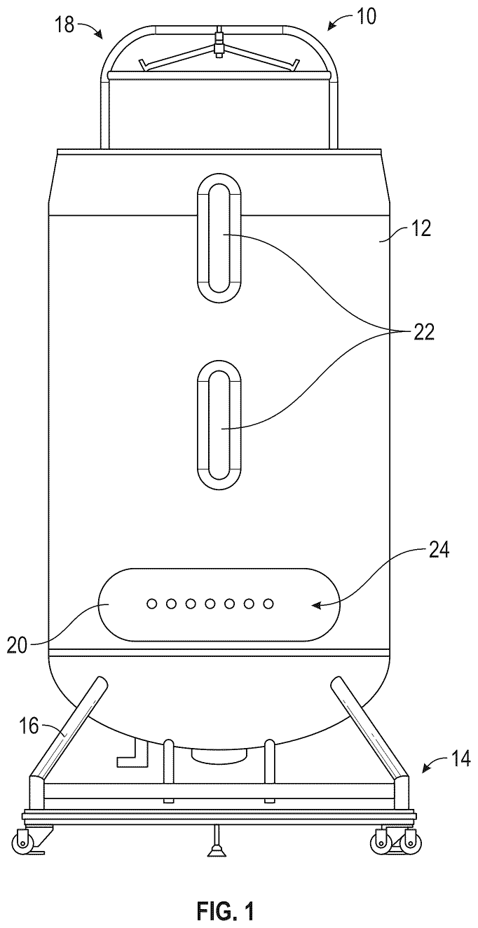

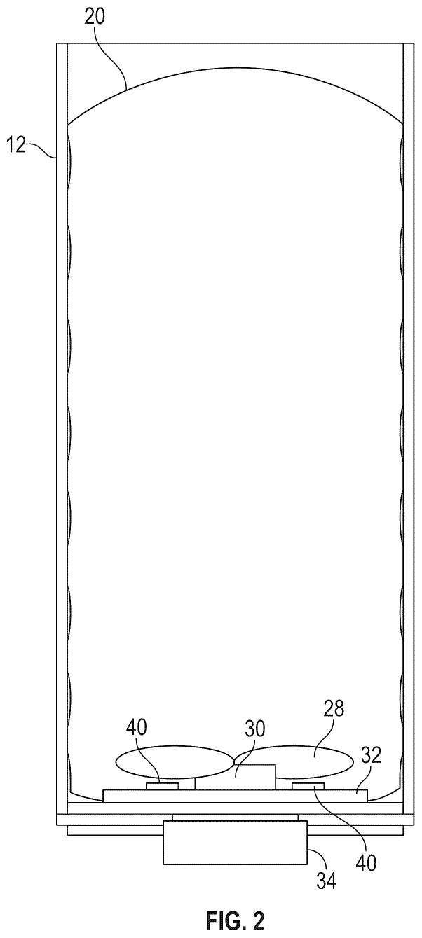

With reference to FIGS. 1 and 2, a bioprocessing system 10 according to an embodiment of the invention is illustrated. The bioprocessing system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 may be outfitted with a lift assembly 18 that provides support to a single-use, flexible bag 20 disposed within the vessel 12. The vessel 12 can be any shape or size as long as it is capable of supporting a single-use flexible bioreactor bag 20. For example, according to one embodiment of the invention the vessel 12 is capable of accepting and supporting a 10-2000 L flexible or collapsible bioprocess bag assembly 20.

The vessel 12 may include one or more sight windows 22, which allows one to view a fluid level within the flexible bag 20, as well as a window 24 positioned at a lower area of the vessel 12. The window 24 allows access to the interior of the vessel 12 for insertion and positioning of various sensors and probes (not shown) within the flexible bag 20, and for connecting one or more fluid lines to the flexible bag 20 for fluids, gases, and the like, to be added or withdrawn from the flexible bag 20. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example.

With specific reference to FIG. 2, a schematic side elevational, cutaway view of the bioprocessing system 10 is illustrated. As shown therein, the single-use, flexible bag 20 is disposed within the vessel 12 and restrained thereby. In embodiments, the single-use, flexible bag 20 is formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem™. Bioclear™ 10 and Bioclear™ 11 laminates, available from Cytiva. Portions of the flexible container can comprise a substantially rigid material such as a rigid polymer, for example, high density polyethylene, metal, or glass. The flexible bag may be supplied pre-sterilized, such as using gamma irradiation.

The flexible bag 20 contains an impeller 28 attached to a magnetic hub 30 at the bottom, center of the inside of the bag, which rotates on an impeller base plate 32 also positioned on the inside bottom of the bag 20. Together, the impeller 28 and hub 30 (and in some embodiments, the impeller plate) form an impeller assembly, however, as user herein, impeller assembly may likewise be used to refer only to the impeller 28. A magnetic drive 34 external to the vessel 12 provides the motive force for rotating the magnetic hub 30 and impeller 28 to mix the contents of the flexible bag 20. While FIG. 2 illustrates the use of a magnetically-driven impeller, other types of impellers and drive systems are also possible, including top-driven and bottom-driven impellers., direct drive impellers, and the like.

As also illustrated in FIG. 2, the bottom of the flexible bag 20 includes one or more sparger elements 40 (also referred to herein as sparger devices or sparge pods). In an embodiment, the sparger elements 40 are affixed to and supported by the impeller base plate 32, although the sparger elements 40 may be affixed to independent sparger base plates that are separate from the impeller base plate 32. The sparger elements 40 are configured for connection to a supply of gas via a port in the bottom or sidewall of the flexible bag 20 and tubing extending form the port to the sparger elements 40. In an embodiment, one or more of the sparger elements 40 are positioned beneath the impeller 28. As known in the art, the sparger elements 40 are used to introduce a specific gas or air into the fluid within the bag 20 in order to agitate and/or dissolve the air or gas into the fluid.

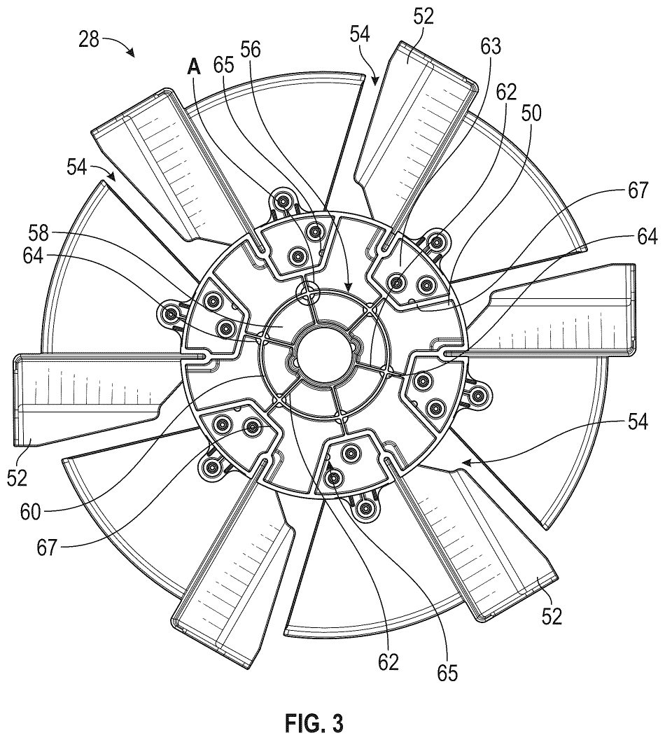

Turning now to FIG. 3, a more detailed view of the impeller 28 according to an embodiment of the invention is shown. The impeller 28 may have any configuration generally known in the art, and includes a central hub 50 and a plurality of blades 52 operatively connected to the hub 50 and extending radially from the hub 50. The hub 50, and thus the impeller 28, are rotatable about a vertical axis (not shown) that extends through the center of the hub 50. In an embodiment, the hub 50 may be a magnetic hub configured to be driven by the magnetic drive system or motor (e.g., motor 34 of FIG. 2) positioned exterior to the flexible bag 20 and vessel 12, as indicated above. As shown therein, the impeller 28 may include a plurality of spaces or voids 54 adjacent to each blade 52, allowing for the passage of fluid therethrough.

As further shown in FIG. 3, an underside of the hub 50 may include an array of ribs 56 that define therebetween recesses or cavities 58. These cavities 58 are locations where gas bubbles from the sparger elements may typically accumulate. The ribs 56 provide reinforcement and/or strengthening of the impeller 28 and its components (including the hub 50). For example, as shown therein, the ribs 56 may include an annular rib 60, and a plurality of radial ribs 62 that intersect the annular rib. Other rib configurations are also possible. In an embodiment, the impeller 28 additionally includes one or more vent holes 64 formed therein that provide pathways for gas bubbles (coming from the sparger elements 40) to travel from an area beneath the impeller 28 to an area above the impeller 28. In an embodiment, the vent holes 64 are located in the hub 50 of the impeller 28. For example, one or more of the vent holes 64 may straddle one of the ribs 56. In an embodiment, one or more of the vent holes 64 may be located at the intersection of ribs (e.g., the intersection of the annular rib 60 and a radial rib 62), as best shown in FIG. 3. In any configuration, the vent holes 64 extend entirely through the impeller 28 so as to provide a fluid pathway through the impeller 28. In an embodiment, every intersection of ribs may include a vent hole so as to provide a fluid pathway through every cavity in the underside of the impeller 28. In an embodiment, there may between 0 and 6 vent holes in the hub 50. In an embodiment, there are three vent holes in the hub 50. In another embodiment, there are six vent holes in the hub 50. In another embodiment, more than six vent holes 50 are present. Other configurations are possible without departing from the broader aspects of the invention. As further shown in FIG. 3, in an embodiment, the underside of the hub 50 may include pockets 63 defined by a rib 67 located in the area where the blades 52 are attached to the hub 50. These pockets 63 may likewise include vent holes 65 allow for passage of gas/fluid from an underside thereof, to the top side thereof.

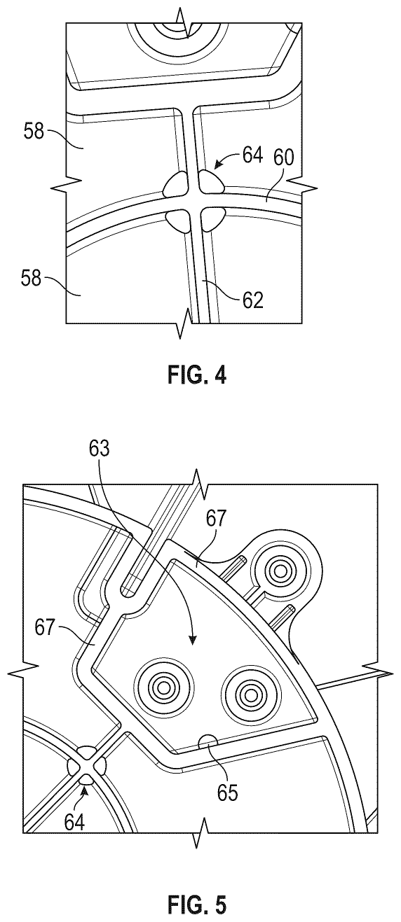

FIG. 4 shows an enlarged, detail view of one of the vent holes 64. As shown therein, by locating the vent hole 64 so as to straddle the ribs, or at the intersection of ribs (e.g., rib 60 and rib 62), a single vent hole can provide venting for a plurality of cavities 58. In an embodiment, the vent holes 64 extend at least partially in a radial direction. FIG. 5 shows and enlarged, detail view of the pockets 63 and vent hole 65 thereof, which allows sparge gas to escape from pocket 63 and pass through to the top side of the impeller 28.

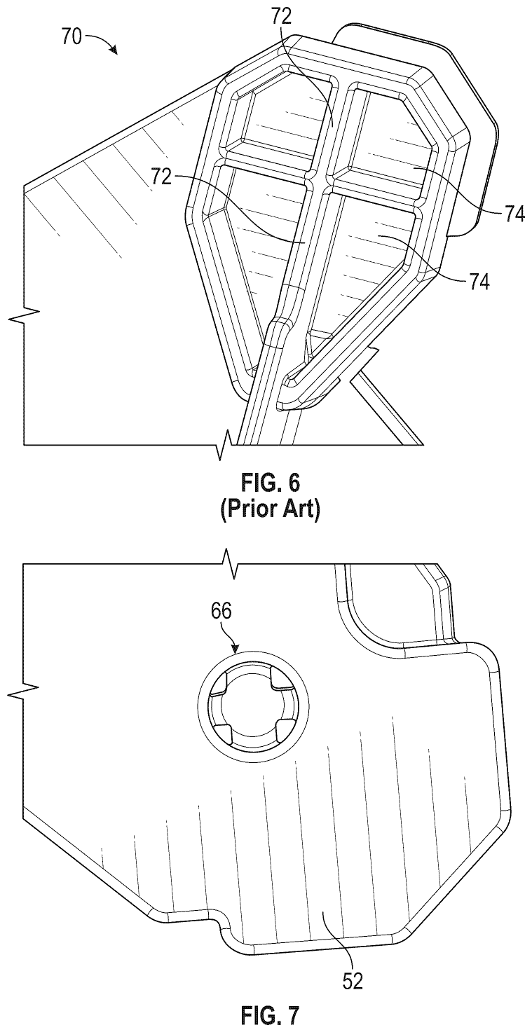

Turning now to FIG. 6, an underside of a blade 70 of a prior art impeller is shown. As illustrated, the blades 70 may include a plurality of ribs 72 defining therebetween cavities 74. Like the ribs of the hub, the ribs on the underside of the blade 70 provide rigidity and strengthening of the blades 70.

In an embodiment, the blades 52 of the impeller 28 of the invention may be similarly configured, namely, with strengthening ribs and cavities on the underside thereof. With reference to FIG. 7, in an embodiment, these blades 52 may likewise include one or more vent holes 66. In an embodiment, the vent holes 66 may straddle the ribs on the underside thereof, or be located at the intersection of ribs. In an embodiment, the vent holes 66 extend at least partially in a radial direction. In an embodiment, each of the blades 52 may include a vent hole 66 therein, although in other embodiments, fewer than all of the blades 52 may include a vent hole 66.

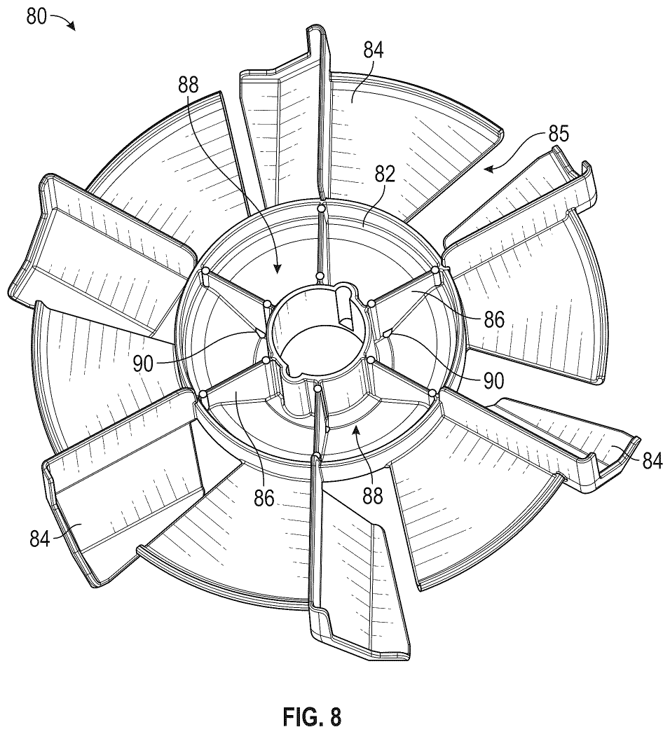

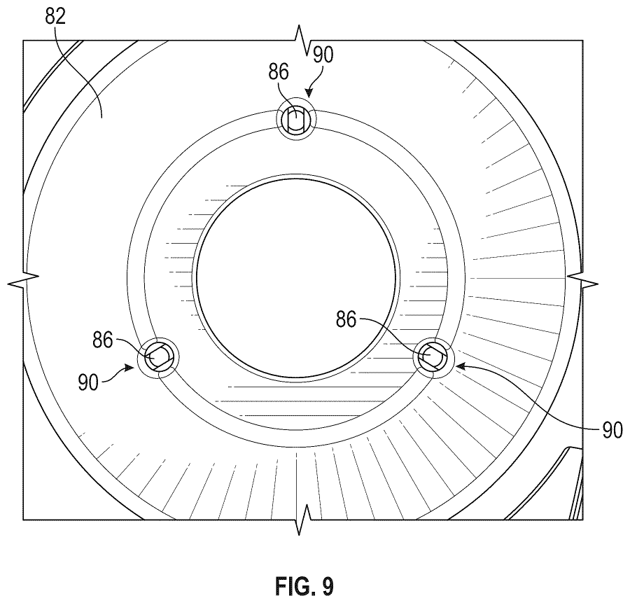

Turning now to FIG. 8, a bottom, perspective view of impeller assembly 80 according to another embodiment of the invention is illustrated. The impeller 80 is generally similar to impeller 28, and includes a central hub 82, and a plurality of blades 84 extending radially from the hub 82. In an embodiment, the central hub 82 may be generally conical or frusto-conical in shape. The blades 84 define voids 85 therebetween, as disclosed above. As further shown therein, the central hub 82 includes a plurality of radial ribs/spokes 86 that define therebetween cavities 88, where sparge gasses could typically accumulate. As shown therein, however, the hub 82 also includes a plurality of vent holes or apertures 90 that provide a pathway for gasses and fluid to travel between an underside of the impeller 80 to a top side of the impeller 80. As best shown in FIG. 9, these vent holes 90 straddles the ribs 86 so that a single vent hole 90 provides venting for two cavities 88. Similar to the embodiments described above, in an embodiment, the vent holes 90 extend at least partially in a radial or lateral direction.

As disclosed above, the impeller assemblies of the invention include one or more vent holes in the hub or blades thereof, which minimizes the possibility that gas bubbles output by sparger elements 40 can rise and become trapped in the cavities in the underside of the hub and/or blades. In particular, the impeller assemblies of the invention provide a fluid pathway so that these rising bubbles can pass through the vent holes and be dispersed throughout the processing volume within the flexible bioprocessing bag 20. Accordingly, the bioprocessing system 10 of the invention, and the impeller 28 or 80 thereof, provides an increased level of gas dispersion and reduced level of cell death due to accumulated bubble popping as compared to existing systems, which increases the efficiency of the bioprocessing system 10 as a whole. Moreover, the vent holes inhibit the trapping of sparge gas within the cavities in the underside of the impeller, reducing impeller vibrations and cavitation.

An impeller assembly for a bioprocessing system includes a hub, a plurality of blades disposed along a circumferential direction of the hub and spaced apart from each other, and at least one vent hole in the impeller assembly providing a pathway for gas to travel from an area beneath the impeller assembly to an area above the impeller assembly. In an embodiment, the at least one vent hole is a plurality of vent holes. In an embodiment, the at least one vent hole is located in the hub of the impeller assembly. In an embodiment, the at least one vent hole is located in at least one of the plurality of blades of the impeller assembly. In an embodiment, the at least one vent hole is a plurality of vent holes, and the plurality of vent holes are located in the hub and the plurality of blades. In an embodiment, the at least one vent hole is a plurality of vent holes, and each blade of the plurality of blades includes at least one vent hole. In an embodiment, at least one of the hub and the plurality of blades includes a rib, and the at least one vent hole straddles the rib. In an embodiment, the plurality of blades each include at least one rib on an underside thereof, the at least one vent hole is a plurality of vent holes, and the plurality of vent holes straddle the at least one rib of the plurality of blades. In an embodiment, the at least one vent hole extends at least partially in a radial direction. In an embodiment, the at least one vent hole is three vent holes. In an embodiment, the at least one vent hole is six vent holes.

According to another embodiment of the invention, a bioprocessing system is provided. The bioprocessing system includes a bioprocessing container, an impeller base plate affixed to a bottom of the bioprocessing container, and an impeller received on the base plate, the impeller including a hub, a plurality of blades disposed along a circumferential direction of the hub and spaced apart from each other, and at least one vent hole in the impeller providing a pathway for gas to travel from an area beneath the impeller to an area above the impeller. In an embodiment the bioprocessing system includes at least one sparger device within the bioprocessing container configured to provide sparge gas to a fluid within the bioprocessing container. In an embodiment, the bioprocessing container is a flexible bag. In an embodiment, the at least one vent hole is a plurality of vent holes. In an embodiment the at least one vent hole is located in the hub of the impeller. In an embodiment the at least one vent hole is located in at least one of the plurality of blades of the impeller. In an embodiment at least one of the hub and the plurality of blades includes a rib, and the at least one vent hole straddles the rib.

According to yet another embodiment of the invention, a method for bioprocessing is provided. The method includes the steps of agitating a fluid within a bioprocessing vessel via rotation of an impeller positioned within the bioprocessing vessel, providing sparge gas to the fluid within the bioprocessing vessel at a location generally beneath the impeller, and via vent holes within the impeller, allowing the sparge gas to pass through the impeller from a location generally beneath the impeller, to a location generally above the impeller. In an embodiment, the vent holes are located in a hub of the impeller.

As used herein, an element or step recited in the singular and proceeded with the word “a” or “an” should be understood as not excluding plural of said elements or steps, unless such exclusion is explicitly stated. Furthermore, references to “one embodiment” of the invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. An impeller assembly for a bioprocessing system, comprising:
a hub;
a plurality of blades disposed along a circumferential direction of the hub and spaced apart from each other; and
at least one vent hole in the impeller assembly providing a pathway for gas to travel from an area beneath the impeller assembly to an area above the impeller assembly.

2. The impeller assembly of claim 1, wherein:
the at least one vent hole is a plurality of vent holes.

3. The impeller assembly of claim 1, wherein:
the at least one vent hole is located in the hub of the impeller assembly.

4. The impeller assembly of claim 1, wherein:
the at least one vent hole is located in at least one of the plurality of blades of the impeller assembly.

5. The impeller assembly of claim 1, wherein:
the at least one vent hole is a plurality of vent holes; and
wherein the plurality of vent holes are located in the hub and the plurality of blades.

6. The impeller assembly of claim 1, wherein:
the at least one vent hole is a plurality of vent holes; and
wherein each blade of the plurality of blades includes at least one vent hole.

7. The impeller assembly of claim 1, wherein:
at least one of the hub and the plurality of blades includes a rib; and
wherein the at least one vent hole straddles the rib.

8. The impeller assembly of claim 1, wherein:
the plurality of blades each include at least one rib on an underside thereof;
wherein the at least one vent hole is a plurality of vent holes; and
wherein the plurality of vent holes straddle the at least one rib of the plurality of blades.

9. The impeller assembly of claim 1, wherein:
the at least one vent hole extends at least partially in a radial direction.

10. The impeller assembly of claim 1, wherein:
the at least one vent hole is three vent holes.

11. The impeller assembly of claim 1, wherein:
the at least one vent hole is six vent holes.

12. A bioprocessing system, comprising:
a bioprocessing container;
an impeller base plate affixed to a bottom of the bioprocessing container; and
an impeller received on the base plate, the impeller including a hub, a plurality of blades disposed along a circumferential direction of the hub and spaced apart from each other, and at least one vent hole in the impeller providing a pathway for gas to travel from an area beneath the impeller to an area above the impeller.

13. The bioprocessing system of claim 12, further comprising:
at least one sparger device within the bioprocessing container configured to provide sparge gas to a fluid within the bioprocessing container.

14. The bioprocessing system of claim 12, wherein:
the bioprocessing container is a flexible bag.

15. The bioprocessing system of claim 12, wherein:
the at least one vent hole is a plurality of vent holes.

16. The bioprocessing system of claim 12, wherein:
the at least one vent hole is located in the hub of the impeller.

17. The bioprocessing system of claim 12, wherein:
the at least one vent hole is located in at least one of the plurality of blades of the impeller.

18. The bioprocessing system of claim 12, wherein:
at least one of the hub and the plurality of blades includes a rib; and
wherein the at least one vent hole straddles the rib.

19. A method for bioprocessing, comprising the steps of:
agitating a fluid within a bioprocessing vessel via rotation of an impeller positioned within the bioprocessing vessel;
providing sparge gas to the fluid within the bioprocessing vessel at a location generally beneath the impeller; and
via vent holes within the impeller, allowing the sparge gas to pass through the impeller from a location generally beneath the impeller, to a location generally above the impeller.

20. The method according to claim 19, wherein:
the vent holes are located in a hub of the impeller.
